(19) United States
(12) Patent Application Publication
(10) Pub. No.: US 2005/0153401 A1
(43) Pub. Date: Jul. 14, 2005

(54) Process for preparing optically active beta-aminocarboxylic acids from racemic n-acylated beta-aminocarboxylic acids

(76) Inventors: Harald Groger, Hanau (DE); Harald Trauthwein, Munchen (DE); Karlheinz Drauz, Freigericht (DE); Stefan Buchholz, Hanau (DE); Christiane Sacherer, Kelkheim (DE); Helge Werner, Bruchkobel (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/508,088

(22) PCT Filed: Mar. 7, 2003
(86) PCT No.: PCT/EP03/02336

(30) Foreign Application Priority Data
Mar. 23, 2002 (DE) 102 13 184.8
Oct. 2, 2002 (DE) 102 46 156.2

(51) Int. Cl. C12P 13/04

(52) U.S. Cl. 435/106; 562/401

(57) ABSTRACT

A process is described for preparing optically active β-atninocarboxylic acids from racemic N-acylated β-aminocarboxylic acids by cnantiosclccthc hydrolysis of the N-acylated β-aminocarboxylic acid in the presence of a hydrolase by way of biocatalyst, wherein the N-acyl substituent of the N-acylated β-aminocarboxylic acid (I) exhibits Structure I in which R¹, R² are each selected, independently of one another, from H, halogen, alkiyl residues, OH, alkoxy residues and aryloxy residues; R³ is selected from halogen, alkoxy residues and aryloxy residues; (II) Structure IIA or IIB or the structure of the corresponding salts or (III) Structure III or the structure of the corresponding salt.

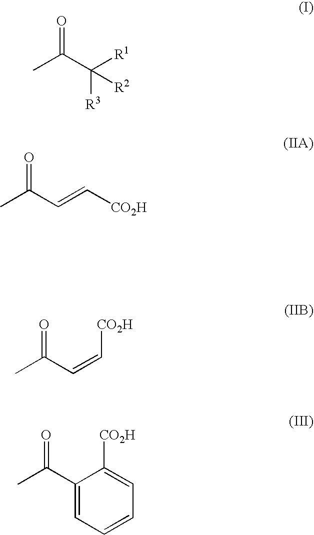

The invention relates to a process for preparing optically active β-aminocarboxylic acids.

Optically active β-aminocarboxylic acids occur in natural substances such as alkaloids and antibiotics, and their isolation is increasingly attracting interest, not least on account of their increasing importance as essential intermediate products in the preparation of medicaments (see, inter alia: E. Juaristi, H. Lopez-Ruiz, Curr. Med. Chem. 1999, 6, 983-1004). Both the free form of optically active β-aminocarboxylic acids and their derivatives show interesting pharmacological effects and can also be employed in the synthesis of modified peptides.

Until now the classical resolution of racemates via diastereomeric salts (proposed route in: H. Boesch et al., Org. Proc. Res. Developm. 2001, 5, 23-27) and, in particular, the diastereoselective addition of lithium phenylethylamide (A. F. Abdel-Magid, J. H. Cohen, C. A. Maryanoff, Curr. Med. Chem. 1999, 6, 955-970) have been established as methods for the preparation of β-aminocarboxylic acids. The latter method is regarded as having been intensively researched and is preferentially adopted, despite numerous disadvantages that arise in the process. On the one hand, stoichiometric quantities of a chiral reagent are required, which represents a great disadvantage in comparison with catalytic asymmetrical methods. Furthermore, expensive and, moreover, hazardous auxiliary substances such as, for example, n-butyllithium are required for activating the stoichiometric reagent by deprotonation. For sufficient stereoselectivity, in addition, the implementation of the reaction at low temperatures of about −70° C. is important, which signifies a high demand on the material of the reactor, additional costs and a high consumption of energy.

Although the preparation of optically active β-aminocarboxylic acids by biocatalytic means plays only a subordinate role at the present time, it is desirable in particular by reason of the economic and ecological advantages of biocatalytic reactions. The use of stoichiometric quantities of a chiral reagent is dispensed with, and small, catalytic quantities of enzymes, which constitute natural and environmentally friendly catalysts, are employed instead. In addition, these biocatalysts which are employed efficiently in the aqueous medium have, besides their catalytic properties and their high effectiveness, the advantage—in contrast with a large number of synthetic metalliferous catalysts—that the use of metalliferous feed materials, particularly those which contain heavy metals and which are consequently toxic, can be dispensed with.

In the state of the art there have already been numerous accounts of, for example, the enantioselective N-acylation of β-aminocarboxylic acids.

For instance, L. T. Kanerva et al. in Tetrahedron: Asymmetry, Vol. 7, No. 6, pp. 1707-1716, 1996 describe the enantioselective N-acylation of ethyl esters of various alicyclic β-aminocarboxylic acids with 2,2,2-trifluoroethyl ester in organic solvents and lipase SP 526 derived from Candida antarctica or lipase PS derived from Pseudomonas cepacia by way of biocatalyst.

V. M. Sánchez et al. investigated the biocatalytic resolution of racemates of (±)-ethyl-3-aminobutyrate (Tetrahedron: Asymmetry, Vol. 8, No. 1, pp. 37-40, 1997) with lipase derived from Candida antarctica via the preparation of N-acetylated β-aminocarboxylic ester.

In EP-A-0 890 649 a process is disclosed for preparing optically active amino esters from racemic amino esters by enantioselective acylation with a carboxylic ester in the presence of a hydrolase, selected from the group comprising amidase, protease, esterase and lipase, and subsequent isolation of the unconverted enantiomer of the amino ester. WO-A-98/50575 relates to a process for obtaining a chiral β-aminocarboxylic acid or its corresponding ester by bringing a racemic β-aminocarboxylic acid, an acyl donor and penicillin G acylase into contact under conditions for acylating an enantiomer of the racemic β-aminocarboxylic acid stereoselectively, the other enantiomer being substantially unconverted, thereby obtaining a chiral β-aminocarboxylic acid.

Desirable, in particular, would be the application to β-aminocarboxylic acids of a biocatalysis technology that is already practised industrially in the case of the α-aminocarboxylic acids. Of interest, above all, is the resolution of racemates of racemic N-acetyl-α-aminocarboxylic acids or corresponding derivatives substituted on the N-acetyl group via enzymatic deacetylation using hydrolases, in particular acylases. The racemic starting compounds can easily be prepared with the aid of acetic-acid derivatives, and their synthesis is, in addition, possible in situ, so the N-acetylated products can be employed directly in the biocatalytic reaction without an additional isolation step. The yields of acetylation reactions are in the quantitative range, and the starting compounds, for instance chloroacetic acid, methoxyacetic acid or acetic anhydride, are inexpensive chemicals which are available in large quantities. A further advantage of such acetyl derivatives in comparison with other acyl derivatives is the easy separability of the N-acetylaminocarboxylic acid from the acetic acid (or the substituted derivatives thereof) after the reaction.

However, until now the application of this concept in respect of β-aminocarboxylic acids has failed. Unfortunately, it has turned out that hydrolases, particularly acylases, do not appear to be suitable for reactions of such a type. H. K. Chenault, J. Dahmer, G. M. Whitesides, J. Am. Chem. Soc. 1989, 111, 6354-6364, established that neither acyclic nor cyclic N-acyl-β-aminocarboxylic acids are suitable as substrates. With regard to the acyclic compound, an N-acetyl compound was investigated. This result has been confirmed by the inventors' own experiments with other hydrolases, in particular with acylases.

Until now there have been accounts only of the enantioselective hydrolysis of racemic N-phenylacetyl-β-aminocarboxylic acids with penicillin acylase (V. A. Soloshonok, V. K. Svedas, V. P. Kukhar, A. G. Kirilenko, A. V. Rybakova, V. A. Solodenko, N. A. Fokina, O. V. Kogut, I. Y. Galaev, E. V. Kozlova, I. P. Shishkina, S. V. Galushko, Synlett 1993, 339-341; V. Soloshonok, A. G. Kirilenko, N. A. Fokina, I. P. Shishkina, S. V. Galushko, V. P. Kukhar, V. K. Svedas, E. V. Kozlova, Tetrahedron: Asymmetry 1994, 5, 1119-1126; V. Soloshonok, N. A. Fokina, A. V. Rybakova, I. P. Shishkina, S. V. Galushko, A. E. Sochorinsky, V. P. Kukhar, M. V. Savchenko, V. K. Svedas, Tetrahedron: Asymmetry 1995, 6, 1601-1610; G. Cardillo, A. Tolomelli, C. Tomasini, Eur. J. Org. Chem. 1999, 155-161). A disadvantage with this process is the difficult reprocessing of the product mixture after the enantioselective hydrolysis. After separation of the free β-aminocarboxylic acid, a mixture is obtained consisting of phenylacetic acid and N-phenylacetyl-β-aminocarboxylic acid, which is difficult to resolve.

Now the object underlying the present invention is to make available a new, simply and economically practicable process for preparing optically active β-aminocarboxylic acids.

This object is achieved, surprisingly, by a process for preparing optically active β-aminocarboxylic acids from racemic N-acylated β-aminocarboxylic acids by enantioselective hydrolysis of the N-acylated -βaminocarboxylic acid in the presence of a hydrolase by way of biocatalyst, wherein the N-acyl substituent of the N-acylated β-aminocarboxylic acid exhibits
(I) Structure I 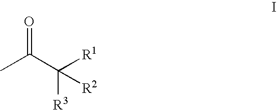
in which R¹, R² are each selected, independently of one another, from H; halogen, preferably chlorine, bromine and fluorine; alkyl residues with preferably 1 to 10 C atoms, in particular methyl, ethyl, n-propyl, isopropyl, n-butyl and tert-butyl; OH; alkoxy residues with preferably 1 to 10 C atoms, in particular methoxy and ethoxy, and aryloxy residues with preferably 6 to 14 C atoms, in particular phenoxy; and
R³ is selected from halogen, preferably chlorine, alkoxy residues with preferably 1 to 10 C atoms, in particular methoxy, and aryloxy residues with preferably 6 to 14 C atoms, in particular phenoxy;
   (II) Structure IIA or IIB 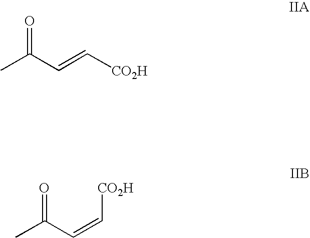
   or the structure of the corresponding salts or
   (III) Structure III 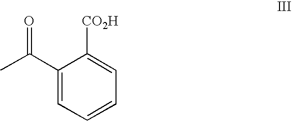
   or the structure of the corresponding salt.

Contrary to previous findings available from the literature and the inventors' own research results, quite unexpectedly a reaction of the special N-acylated β-aminocarboxylic acids with a hydrolase takes place.

The enantioselective hydrolysis proceeds in particularly effective manner with an N-acyl substituent having Structure I if R³ is chlorine, where appropriate R¹ or R² is also chlorine, or if R³ is methoxy or if R¹, R² and R³ are each fluorine. Exemplary N-acyl substituents are N-chloroacetyl, N-dichloroacetyl, N-methoxyacetyl and N-trifluoroacetyl. A further advantage of these N-acyl substituents is that the acetic-acid derivatives arising therefrom in the course of hydrolysis can easily be separated from the product mixture on account of their relatively low molecular weight.

In particular, the process is suitable for preparing optically active aromatic β-aminocarboxylic acids by conversion of an N-acylated β-aminocarboxylic acid having the following structure IV, 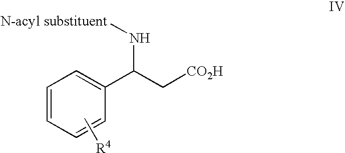
in which the N-acyl substituent is defined as previously; R⁴ is selected from H; alkyl residues with preferably 1 to 10 C atoms, in particular methyl, ethyl, propyl and butyl; OH, alkoxy residues with preferably 1 to 4 C atoms, in particular methoxy and ethdxy; and halogen. It is a particular advantage if the N-acyl substituent exhibits Structure I from Claim 1, in which R¹ and R² are each H and R³ is chlorine, and R⁴ is equal to H.

The process according to the invention is particularly suitable for preparing optically active 3-amino-3-phenylpropionic acid (β-amino-β-phenylpropionic acid) from the corresponding racemic N-acylated 3-amino-3-phenylpropionic acid.

The process according to the invention is also advantageous for preparing optically active aliphatic β-aminocarboxylic acids by conversion of an N-acylated β-aminocarboxylic acid having the following Structure V, 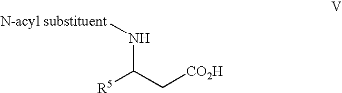
in which R⁵ stands for an alkyl group, in particular a methyl, ethyl, n-propyl, isopropyl, n-butyl or tert-butyl group, or a substituted alkyl group, in particular a substituted methyl, ethyl, n-propyl, isopropyl, n-butyl or tert-butyl group. The substituents are preferably selected from halogens, benzyl and N—, O— and S-containing substituents.

The racemic N-acylated β-aminocarboxylic acids employed as starting compounds are generally obtained from the racemic β-aminocarboxylic acids by conversion with suitable acid chlorides or anhydrides. Also possible are the preparation of the racemic N-acylated β-aminocarboxylic acids in situ and their direct use in the biocatalytic reaction.

In the process according to the invention a large number of enzymes can be employed as hydrolases; suitable hydrolases are, for example, acylases, proteases, lipases or esterases, preferably acylases. The use of pig-kidney acylase of type I has proved particularly suitable. But the reaction is also possible by using a protease, preferably derived from Aspergillus, and more preferably from Aspergillus oryzae.

The enzyme that is used can be employed in native or immobilised form. The use of genetically engineered enzymes is also possible.

The process according to the invention is preferably implemented in aqueous solution. The pH value is usually between 6 and 10, preferably between 7 and 9.

In aqueous solution the concentration of the N-acylated β-aminocarboxylic acid preferably amounts to 2 to 40 wt. %, more preferably 5 to 20 wt. %, relative to the total quantity in the reaction mixture.

Besides being carried out in aqueous solution, the process according to the invention can also be carried out in organic solvents, preferably in water-miscible solvents such as methanol and ethanol for instance, and also in appropriate mixtures of organic solvents with water.

The quantity of enzyme to be added depends on the type of the hydrolase and the activity of the enzyme preparation. The optimal quantity of enzyme for the reaction can easily be ascertained by a person skilled in the art by simple preliminary tests.

The hydrolysis of the N-acylated β-aminocarboxylic acid under enzyme catalysis is ordinarily carried out at temperatures between 10° C. and 60° C., in particular between 20° C. and 40° C.

The progress of the reaction can easily be observed by conventional methods, for example by means of HPLC. The resolution of racemates is sensibly brought to an end at a conversion of 50% of the racemic N-acylated β-aminocarboxylic acid. This is done, as a rule, by removing the enzyme from the reaction chamber, for example by filtration, preferably ultrafiltration.

As a result of the enantioselective hydrolysis of the racemic N-acylated β-aminocarboxylic acid, the corresponding β-aminocarboxylic acid arises from the one enantiomer, whereas the other enantiomer is substantially unconverted. The mixture that is now present, consisting of N-acylated β-aminocarboxylic acid and β-aminocarboxylic acid, can easily be separated by conventional methods. Well-suited for the separation of the mixture are, for example, extraction and/or filtration processes at suitable pH values.

It is possible for the process according to the invention to be made still more economical if, after separation of the desired enantiomer, the remaining, unwanted enantiomer is racemised in accordance with methods known in the state of the art and is reintroduced into the process.

As a result of this recycling, it becomes possible to obtain a total of more than 50% of the desired enantiomer from the racemic N-acylated β-aminocarboxylic acid.

The process according to the invention is not only suitable for preparing optically active β-aminocarboxylic acids but may also be part of complicated multistage syntheses, for example in connection with the preparation of medicaments or crop-protection agents.

The invention will now be illustrated on the basis of the following Examples.

### EXAMPLE 1

### COMPARATIVE EXAMPLE

In a reaction vessel a solution consisting of 900 μl of a 50 mM sodium-phosphate buffer with pH=8.0, 100 μl of a 0.1 M aqueous solution of rac-N-acetyl-3-amino-3-phenylpropionic acid and 5 mg pig-kidney acylase of type I (producer: Sigma) is stirred at 30° C. for 24 h and subsequently the conversion-rate is determined by means of HPLC (column: Nautilus; eluent: H₂O and acetonitrile in a volume ratio of 80:20 with 0.1 vol. % trifluoroacetic acid, 220 nm, 1 ml/min; injection: 900 μl eluent+100 μl reaction mixture). The conversion-rate is <1%.

### EXAMPLE 2

In a reaction vessel a solution consisting of 950 μl of a 50 MM sodium-phosphate buffer with pH=8.0, 50 μl of a 10% (w/vol. %) solution of rac-N-chloroacetyl-3-amino-3-phenylpropionic acid in acetone and 5 mg pig-kidney acylase of type I (producer: Sigma) is stirred at 30° C. for 24 h and subsequently the conversion-rate is determined by means of HPLC (column: Nautilus; eluent: H₂O and acetonitrile in a volume ratio of 80:20 with 0.1 vol. % trifluoroacetic acid, 220 nm, 1 ml/min; injection: 900 μl eluent+100 μl reaction mixture). The conversion-rate is 14%.

### EXAMPLE 3

In a reaction vessel 50 ml of an aqueous solution consisting of a potassium-phosphate buffer with pH=7.0 and also 127 mg rac-N-chloroacetyl-3-amino-3-phenylpropionic acid (0.5 mmol) are charged. Subsequently 120 mg of the pig-kidney acylase of type I (producer: Sigma) are added and the reaction mixture is allowed to react at room temperature (about 25° C.). The conversion after five days is 9%, and after 19 days 46% (according to HPLC of the reaction sample).

### EXAMPLE 4

In a reaction vessel 50 ml of an aqueous solution of 127 mg rac-N-chloroacetyl-3-amino-3-phenylpropionic acid (0.5 mmol), which was adjusted by means of NaOH to pH=8.2, are charged and brought to a temperature of 30° C. Subsequently 120 mg of the pig-kidney acylase of type I (producer: Sigma) are added and the reaction mixture is allowed to react at a reaction temperature of 30° C. After five days the conversion is 24% (according to HPLC of the reaction sample). After a period of 13 days the reaction mixture is firstly separated from the enzyme component by ultrafiltration. A clear filtrate is obtained, from which the conversion-rate and also the enantioselectivity with respect to the optically active 3-amino-3-phenylpropionic acid that has been formed are then determined. The conversion-rate is 35%, and ee values >98% were ascertained for the enantioselectivity.

### EXAMPLE 5

In a reaction vessel a solution consisting of 950 μl of a 50 mM sodium-phosphate buffer with pH=7.5, 50 μl of a 10% (w/vol. %) solution of rac-N-chloroacetyl-3-amino-3-phenylpropionic acid in acetone, and 5 mg protease derived from Aspergillus oryzae (producer: Sigma: protease XXIII) are stirred at 30° C. for four days and subsequently the conversion-rate is determined by means of HPLC as in Example 2. The conversion-rate is 6%.

### EXAMPLE 6

### Optimnised preparation of (S)-3-amino-3-(phenyl)propionic acid

In a 100 mL reaction vessel 60.4 mg rac-N-chloroacetyl-3-amino-3-phenylpropionic acid (purity: >98%; 0.25 mmol) are added to 12.5 ml water and adjusted with NaOH to a pH value of pH 7.75. After this, 2.5 mL of a 0.001 M cobalt(II)-chloride solution are added, topping-up is effected with 12.5 mL of a buffer solution (50 mM phosphate buffer), and the solution that has arisen is brought to a temperature of 37.5° C. Subsequently 60 mg of the pig-kidney acylase of type I (producer: Sigma) are added and the reaction mixture is allowed to react at a reaction temperature of 37.5° C. After one day the conversion is 43.2%, and after two days 48.7% (according to HPLC of the reaction sample). After this, the reaction mixture is separated from the enzyme component by ultrafiltration. A clear filtrate is obtained, from which the enantioselectivity with respect to the optically active (S)-3-amino-3-phenylpropionic acid that has been formed is then determined. For the enantioselectivity, ee values of 99.0% were ascertained.

### EXAMPLE 7

### Optimised preparation of optically active 3-amino-3-(2-thiophenyl)propionic acid

In a 100 mL reaction vessel 63 mg rac-N-chloroacetyl-3-amino-3-(2-thienyl)propionic acid (purity: 98.3%; 0.25 mmol) are added to 12.5 ml water and adjusted with NaOH to a pH value of pH 7.75. After this, 2.5 mL of a 0.001 M cobalt(II)-chloride solution are added, topping-up is effected with 12.5 mL of a buffer solution (50 mM phosphate buffer), and the solution that has arisen is brought to a temperature of 37.5° C. Subsequently 60 mg of the pig-kidney acylase of type I (producer: Sigma) are added and the reaction mixture is allowed to react at a reaction temperature of 37.5° C. After one day the conversion is 49.2%, and after two days 50.0% (according to HPLC of the reaction sample). After this, the reaction mixture is separated from the enzyme component by ultrafiltration. A clear filtrate is obtained, from which the enantioselectivity with respect to the optically active 3-amino-3-(2-thienyl)propionic acid that has been formed is then determined. For the enantioselectivity, ee values >99.0% were ascertained.

### EXAMPLE 8

### Optimised preparation of optically active 3-amino-3-(p-fluorophenyl)propionic acid

In a 100 mL reaction vessel 66.1 mg rac-N-chloroacetyl-3-amino-3-(p-fluorophenyl)propionic acid (purity: 98.2%; 0.25 mmol) are added to 12.5 ml water and adjusted with NaOH to a pH value of pH 7.75. After this, 2.5 mL of a 0.001 M cobalt(II)-chloride solution are added, topping-up is effected with 12.5 mL of a buffer solution (50 mM phosphate buffer), and the solution that has arisen is brought to a temperature of 37.5° C. Subsequently 60 mg of the pig-kidney acylase of type I (producer: Sigma) are added and the reaction mixture is allowed to react at a reaction temperature of 37.5° C. After one day the conversion is 32.9%, and after two days 45.6% (according to HPLC of the reaction sample). After this, the reaction mixture is separated from the enzyme component by ultrafiltration. A clear filtrate is obtained, from which the enantioselectivity with respect to the optically active 3-amino-3-(p-fluorophenyl)propionic acid that has been formed is then determined. For the enantioselectivity, ee values >95.0% were ascertained.

1. A process for preparing optically active β-aminocarboxylic acids from racemic N-acylated β-aminocarboxylic acids by enantioselective hydrolysis of the N-acylated β-aminocarboxylic acid in the presence of a hydrolase by way of biocatalyst, wherein the N-acyl substituent of the N-acylated β-aminocarboxylic acid exhibits
(I) Structure I 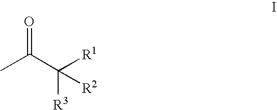
in which R¹, R² are each selected, independently of one another, from H, halogen, alkyl residues, OH, alkoxy residues and aryloxy residues,
R³ is selected from halogen, alkoxy residues and aryloxy residues,
(II) Structure IIA or IIB 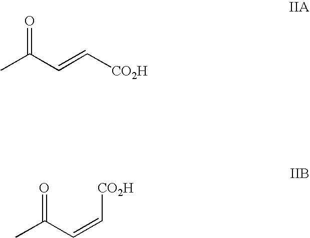
or the structure of the corresponding salts or
(III) Structure III 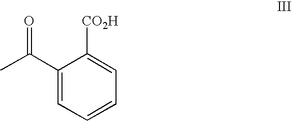
or the structure of the corresponding salt.

2. Process according to claim 1,
wherein
the N-acyl substituent of the N-acylated β-aminocarboxylic acid exhibits Structure I, in which R¹, R² are each selected, independently of one another, from H, chlorine, bromine, fluorine, methyl, ethyl, n-propyl, isopropyl, n-butyl, tert-butyl, methoxy and ethoxy and R³ is selected from chlorine and methoxy.

3. Process according to claim 2,
wherein
R¹ and R² are each H and R³ is chlorine.

4. Process according to claim 2,
wherein
R¹ is equal to H and R² and R³ are each chlorine.

5. Process according to claim 2,
wherein
R¹ and R² are each H and R³ is methoxy.

6. Process according to claim 1,
wherein
R¹, R² and R³ are each fluorine.

7. Process according to claim1,
wherein
the N-acylated β-aminocarboxylic acid is an aromatic N-acylated β-aminocarboxylic acid having Structure IV 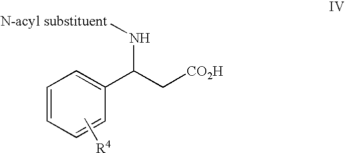
wherein
R⁴ is selected from H, alkyl residues, OH, alkoxy residues, and halogen.

8. Process according to claim 7,
wherein
the N-acyl substituent exhibits Structure I, in which R¹ and R² are each H and R³ is chlorine, and R⁴ is equal to H.

9. The process according to claim 1,
wherein
the N-acylated β-aminocarboxylic acid is an aliphatic N-acylated β-aminocarboxylic acids of the following Structure V, 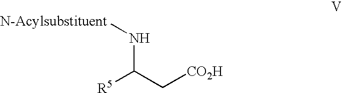
in which R⁵ stands for an alkyl group.

10. Process according to claim 1,
wherein
the hydrolase is an acylase, protease, lipase or esterase.

11. Process according to claim 9,
wherein
the acylase is pig-kidney acylase of type I.

12. Process according to claim 9,
wherein
the hydrolase is a protease derived from Aspergillus.

13. Process according to claim 11,
wherein
the protease is derived from Aspergillus oryzae.
